(19) United States
(12) Patent Application Publication
(10) Pub. No.: US 2006/0205028 A1
(43) Pub. Date: Sep. 14, 2006

(54) Active-site titration of glycosyl hydrolases

(75) Inventors: Lars Lehmann Hylling Christensen, Allerod (DK); Christel Joergensen, Lyngby (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/568,243

(22) PCT Filed: Aug. 19, 2004
(86) PCT No.: PCT/DK04/00548

§ 371 Date: Feb. 14, 2006

(30) Foreign Application Priority Data
Aug. 19, 2003 (DK) PA 2003 01184

(51) Int. Cl. C12Q 1/34

(52) U.S. Cl. 435/18

(57) ABSTRACT

The invention provides a method of screening for a property of a glycosyl hydrolase wherein the property is dependent on the concentration of the glycosyl hydrolase. The method comprises a step for determining the concentration of the glycosyl hydrolase by active site titration.

### BACKGROUND

When libraries of enzymes are screened for properties which are dependent on the concentration of the enzyme, such as specific activity, it is essential to have available a fast and reliable method for determining the concentration of the enzyme. It is an advantage if the method can be implemented in a standard microtiter plate based screening setup. The method must also be fast enough not to be a major bottleneck in high throughput screening. Further it must be capable of determining the concentration of the enzyme based on very small volumes of enzyme solution—e.g. less than the volume of a well in a microtiter plate. The present invention has all the above-mentioned advantages.

### SUMMARY

The present invention discloses a method for determining the concentration of glycosyl hydrolases, which can form part of a screening setup. Accordingly, as a first aspect, the invention provides a method for determining the concentration of a glycosyl hydrolase by active-site titration using an inhibitor having a K_{d} which is at least 25 times lower than the concentration of glycosyl hydrolase or, when the glycosyl hydrolase is a retaining glycosyl hydrolase, using a substrate wherein the rate constant for the glycosylation step is at least 10 times larger than for the deglycosylation step.

In a second aspect, the invention provides a method of screening for a property of a glycosyl hydrolase wherein the property is dependent on the concentration of the glycosyl hydrolase, comprising the steps of:
a) arranging a population of host cells expressing glycosyl hydrolases in a spatial array wherein each position of the spatial array is occupied by one or more cells expressing a specific glycosyl hydrolase,
b) cultivating the host cells in a suitable growth medium,
c) determining the concentration of the glycosyl hydrolase of each position of the spatial array by active-site titration using an inhibitor having a K_{d} which is at least 25 times lower than the concentration of glycosyl hydrolase or, when the glycosyl hydrolase is a retaining glycosyl hydrolase, using a substrate wherein the rate constant for the glycosylation step is at least 10 times larger than for the deglycosylation step,
d) assaying the glycosyl hydrolase of each position of the spatial array for the property and relating the result to the concentration.

### DETAILED DESCRIPTION

Active Site Titration Using Tight-Binding Inhibitor

Active concentration of an enzyme can be determined if a suitable inhibitor is available. The inhibitor should react with the enzyme with a known stochiometric ratio, preferably 1:1. The inhibitor-enzyme complex should have reduced activity compared to uncomplexed enzyme with a given substrate; preferably the inhibitor-enzyme complex should be inactive.

The affinity of the inhibitor for the enzyme should be sufficiently strong to assure that only insignificant amount of free inhibitor is present when inhibitor is mixed with a surplus of enzyme, i.e. for active site titration to be applicable, the dissociation constant Kd for the reaction:
E+I←→EI
where E is free enzyme, I is free inhibitor, EI is the enzyme-inhibitor complex, and K_{d}=[E]*[I]/[EI] at equilibrium, should be at least 25 times, preferably at least 50 times, more preferably at least 100 times, most preferably at least 500 times, and in particular at least 1000 times lower than the enzyme concentrations used. This requirement of course includes inhibitors where reaction with enzyme is irreversible.

The inhibitor should be specific, i.e. binding of inhibitor to other compounds in the enzyme solution should be insignificant (i.e. either the concentration of these other compounds with reactivity towards/affinity for the inhibitor should be much lower than the enzyme concentration or their reactivity towards/affinity for the inhibitor should be low).

Normally, active site titration of an enzyme solution will be done by mixing at least two (preferably more) aliquots of the enzyme solution with various suitable amounts of inhibitor. The mixtures of inhibitor and enzyme are incubated under conditions assuring that reaction between inhibitor and enzyme gets sufficiently close to equilibrium. At least two (preferably more) inhibitor concentrations below the equivalence point with enzyme should be used. Subsequently, residual enzyme activities in the inhibitor/enzyme mixtures are measured using a suitable substrate. Preferably, the substrate should be unable to affect the equilibrium between inhibitor and enzyme significantly. This can e.g. be accomplished by using the substrate at concentrations much lower than the Michaelis-Menten constant Kₘ or by assuring that the incubation time with substrate is short compared to the dissociation rate for the enzyme-inhibitor complex.

Active Site Titration Using Burst Titration with Specific Substrate

Hydrolysis by retaining glycosyl hydrolases can be described by the reaction scheme: 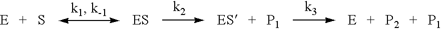
where E is the enzyme, S is the substrate, ES′ is a glycosyl-enzyme intermediate formed by the glycosylation step, P₁ is the product released in the glycosylation step corresponding to the fragment with the newly formed non-reducing end or the aglycon, P₂ is the product released in the deglycosylation step corresponding to the fragment with the newly formed reducing end, and k₁, k₋₁, k₂ and k₃ are reaction rate constants.

Assuming quasi steady-state for the concentration of the intermediate ES and that the used substrate concentration [S] is much larger than the total enzyme concentration [E]ₜₒₜ and therefore approximately constant during the experiment, integration of the differential equation for the formation of product P₁ gives:

$$[ {P}_{1} ] = \frac{\frac{{k}_{2} \cdot {k}_{3}}{{k}_{2} + {k}_{3}} \cdot [ S ] \cdot {[ E ]}_{tot}}{{K}_{M} + [ S ]} \cdot t + {( \frac{\frac{{k}_{2}}{{k}_{2} + {k}_{3}}}{1 + \frac{{K}_{M}}{[ S ]}} )}^{2} \cdot {[ E ]}_{tot} \cdot ( 1 - {ⅇ}^{- ( \frac{{K}_{M} \cdot [ S ]}{\frac{{K}_{M}}{{k}_{3}} + \frac{[ S ]}{{k}_{2} + {k}_{3}}} ) \cdot t} )$$

where K_{M} is the observed Michaelis-Menten constant when quasi-steady state is reached for the intermediate ES′ and given by:

$${K}_{M} = \frac{( {k}_{- 1} + {k}_{2} ) \cdot {k}_{3}}{{k}_{1} \cdot ( {k}_{2} + {k}_{3} )}$$

As seen from the equation for formation of P₁, the initial burst will be followed by a phase where formation of P₁ is approximately linear and given by:

$$[ {P}_{1} ] = \frac{\frac{{k}_{2} \cdot {k}_{3}}{{k}_{2} + {k}_{3}} \cdot [ S ] \cdot {[ E ]}_{tot}}{{K}_{M} + [ S ]} \cdot t + {( \frac{\frac{{k}_{2}}{{k}_{2} + {k}_{3}}}{1 + \frac{{K}_{M}}{[ S ]}} )}^{2} \cdot {[ E ]}_{tot} = α \cdot t + β$$

The intercept β is seen to be approximately equal to the total enzyme concentration [E]ₜₒₜ if the rate constant k₂ is much larger than the rate constant k₃ and the substrate concentration [S] is much larger than the Michaelis-Menten constant K_{M}.

Thus, burst titration requires a substrate where the rate constant for the glycosylation step k₂ is at least 10 times, preferably at least 50 times, more preferably at least 100 times, most preferably at least 500 times, and in particular at least 1000 times larger than the rate constant for the deglycosylation step k₃ and the product P₁ is detectable. The substrate concentration should be at least 10 times, preferably at least 100 times the concentration of the enzyme. Also, the substrate concentration should preferably be at least 10 times the Michaelis-Menten constant K_{M}, otherwise the release of P₁ should be measured with at least two different substrate concentrations. With these requirements fulfilled, the total enzyme concentration [E]ₜₒₜ can be found by fitting measured concentrations of P₁ to the equations above.

Inhibitors/Substrates

One class of inhibitors according to the invention, which are suitable for determining the concentration of glucoamylases (and other alpha-glucosidases) comprise acarbose and homologous thereof. All these pseudo-oligosaccharide inhibitors have an acarviosine moiety at the non-reducing end with various sugars attached to the reducing end. In acarbose, maltose is attached to the acarviosine. The resemblance of the planar structure of the hydroxymethylconduritol unit at the non-reducing end of acarbose to the transition state for hydrolysis of maltodextrins results in tight binding to the active site of glucoamylase, and the low reactivity of the N-glucosidic linkage between the hydroxymethylconduritol residue and the 4,6-dideoxy-4-amino-D-glucopyranose residue assures that the acarbose in not hydrolysed.

Other examples of inhibitors which may successfully be used in the present invention, include, but are not limited to:

tendamistat,

trestatin,

oligostatin,

nojirimycin and 1-deoxy-nojirimycin,

pyridinolol,

various isoflavinoids,

panosialin, and

siastatin A and B.

References to most of these inhibitors may be found in Walker J M et al, Applied Biochemistry and Biotechnology 38: 141 (1993).

Still other examples of useful inhibitors are:

BASI (see e.g. Rodenburg-K W et al European Journal of Biochemistry 267 p. 1019 (2000)), and

T-76 alpha-amylase inhibitor (see Sumitani-j Bioscience biotechnology and biochemistry 57: 1243 (1993)).

Glycosyl Hydrolases

The glycosyl hydrolases according to the invention are those enzymes acting on glycosidic bonds, which belong to EC 3.2.-.- (as defined in the Recommendations of the Nomenclature Committee of the International Union of Biochemistry and Molecular Biology on the Nomenclature and Classification of Enzyme-Catalysed Reactions). It should be noted that some of these enzymes are also able to transfer glycosyl residues to oligosaccharides, polysaccharides and other alcoholic acceptors.

Of particular interest for the present invention are enzymes hydrolysing o-glycosyl bonds. These enzymes belong to EC 3.2.1.-. Non-limiting examples of these are:
EC 3.2.1.3 glucan 1,4-alpha-glucosidases, also known as amyloglucosidases or glucoamylases,
EC 3.2.1.20 alpha-glucosidases, and
EC 3.2.1.1 alpha-amylase

An alternative way of classifying enzymes is related to their structure. The CAZy database (see e.g. Davies G., Henrissat B. Structures and mechanisms of glycosyl hydrolases. Structure 3:853-859 (1995); and Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12) describes the families of structurally-related catalytic and carbohydrate-binding modules (or functional domains) of enzymes that degrade, modify, or create glycosidic bonds.

Currently, at least 91 families of Glycosyl hydrolases are described. Of preferred interest for the present invention are the following families:

Family 13, including the following activities: alpha-amylase (EC 3.2.1.1); pullulanase (EC 3.2.1.41); cyclomaltodextrin glucanotransferase (EC 2.4.1.19); cyclomaltodextrinase (EC 3.2.1.54); trehalose-6-phosphate hydrolase (EC 3.2.1.93); oligo-alpha-glucosidase (EC 3.2.1.10); maltogenic amylase (EC 3.2.1.133); neopullulanase (EC 3.2.1.135); alpha-glucosidase (EC 3.2.1.20); maltotetraose-forming alpha-amylase (EC 3.2.1.60); isoamylase (EC 3.2.1.68); glucodextranase (EC 3.2.1.70); maltohexaose-forming alpha-amylase (EC 3.2.1.98); branching enzyme (EC 2.4.1.18); trehalose synthase (EC 5.4.99.16); 4-alpha-glucanotransferase (EC 2.4.1.25); maltopentaose-forming alpha-amylase (EC 3.2.1.-); amylosucrase (EC 2.4.1.4); sucrose phosphorylase (EC 2.4.1.7); malto-oligosyltrehalose trehalohydrolase (EC 2.4.1.141); isomaltulose synthase (EC 5.4.99.11).

Family 14, including beta-amylase (EC 3.2.1.2).

Family 15, including the following activities: glucoamylase (EC 3.2.1.3); glucodextranase (EC 3.2.1.70).

Family 31, including the following activities: alpha-glucosidase (EC 3.2.1.20); glucoamylase (EC 3.2.1.3); sucrase-isomaltase (EC 3.2.1.48) (EC 3.2.1.10); alpha-xylosidase (EC 3.2.1.-); alpha-glucan lyase (EC 4.2.2.13); isomaltosyltransferase (EC 2.4.1.-).

Family 57, including the following activites: alpha-amylase (EC 3.2.1.1); 4-alpha-glucanotransferase (EC 2.4.1.-); alpha-galactosidase (EC 3.2.1.22).

Family 63, including processing alpha-glucosidase (EC 3.2.1.106).

Of more preferred interest are glycosyl hydrolases belonging to families 13 and 15, and most preferably glycosyl hydrolases belonging to family 15.

The glycosyl hydrolases of families 1, 2, 3, 5, 7, 10, 11, 12, 13, 16, 17, 18, 20, 22, 26, 27, 29, 30, 31, 32, 33, 34, 35, 36, 38, 39, 42, 51, 52, 53, 54, 56, 57, 59, 66, 68, 70, 72, 77, 79, 83, 85 and 86 are retaining glycosyl hydrolases.

Screening

Screening of enzymes has been described in e.g. WO 01/32844. The screening method of the invention may be semi or fully automated; it may be referred to as high throughput screening; it may be capable of screening at least 100, preferably at least 500, more preferably at least 1000, most preferably 5000, and in particular at least 10000 glycosyl hydrolases in a continuous operation with no significant human intervention, except for feeding the setup with miscellaneous consumables and removing waste; and it may be capable of screening at least 50, preferably at least 100, more preferably at least 250, most preferably 500, and in particular at least 1000 glycosyl hydrolases in 24 hours.

In the method of the invention, the glycosyl hydrolases are screened for a property which is dependent on the concentration of the enzyme, in other words a specific property, i.e. a property which has been normalized by taking the amount of enzyme protein into account. Examples of specific properties include, but are not limited to, specific activity (such as activity per mg enzyme or activity per mole) and specific performance (such as wash performance).

Relating the Screening Result to the Concentration

When carrying out the screening method of the invention, the concentration of glycosyl hydrolase is determined in step c). The concentration must then be related to the screening result by either:

adjusting the concentration of glycosyl hydrolase in each position to essentially the same level and then performing the assay of step d); or

performing the assay of step d) and then correcting the data obtained with regard to the concentration of the glycosyl hydrolase, based on knowledge of dosage-response kinetics.

The present invention is further described by the following examples which should not be construed as limiting the scope of the invention.

### EXAMPLES

Chemicals used as buffers and substrates were commercial products of at least reagent grade.

### Example 1

Active Site Titration of Glucoamylase Variants and Determination of their Specific Activity

Glucoamylase variants may be prepared as described in Sauer J et al., Biochimica et Biophysica Acta, Vol. 1543 (2), pp. 275-293 (2000) “Glucoamylase: Structure/function relationships, and protein engineering”;

or as described in Frandsen T P et al., “Increasing the thermal stability and catalytic activity of Aspergillus niger glucoamylase by combining site specific mutations and directed evolution”, In Carbohydrate Bioengineering, RS—C eds. T T Teeri, B Svensson, H J Gilbert and T Feizi, Proceedings of the 4th carbohydrate meeting, 2001.

The Talaromyces emersonii glucoamylase is disclosed in WO 99/28448.

A deep well microtiter plate with 32 Talaromyces emersonii glucoamylase variants, each grown in three wells, was centrifuged for 5 min at 3000 rpm. Obtained culture supernatants were transferred to another deep well microtiter plate. From each well eight aliquots of 40 μl culture supernatant were transferred to a microtiter plate and mixed with 20 μl of acarbose diluted to varying concentrations (0, 0.0625, 0.125, 0.25, 0.5, 1.0, 2.0 and 4.0 μM in 0.2 M sodium acetate buffer, 0.01% Triton X-100, pH 4.5). After incubation for 1 hour at room temperature with agitation, 50 μl substrate solution (10 mM p-nitrophenyl-alpha-D-glucose (pNP-Glu) in 0.2 M sodium acetate buffer, 0.01% Triton X-100, pH 4.5) was added. After 2 hours incubation at room temperature with agitation, the reaction was stopped by adding 50 μl sodium carbonate pH 9.5. Absorbance was read at 405 nm using a microtiter plate reader (SpectraMax Plus, Molecular Devices), and concentrations of culture supernatants were determined by linear regression with acarbose concentrations showing residual activity. Highest concentration of acarbose was in all cases able to inhibit all glucoamylase activity. No significant deviations from a straight line were visible for acarbose concentrations below the equivalence point indicating that equilibrium between inhibitor and enzyme was reached and affinity of inhibitor for enzyme was sufficiently high.

An example of measured and fitted absorbances with the variant Var10 is given in Table

| Acarbose concentration | | |
|---|---|---|
| (μM) | A₄₀₅ measured | A₄₀₅ fitted |
| 0 | 2.00 | 2.00 |
| 0.0625 | 2.06 | 1.91 |
| 0.125 | 1.80 | 1.82 |
| 0.25 | 1.57 | 1.64 |
| 0.5 | 1.17 | 1.28 |
| 1 | 0.63 | 0.56 |
| 2 | 0.04 | 0.02 |
| 4 | 0.01 | 0.02 |

Table 2. Measured and fitted absorbances of one of the three deep well fermentations with the variant Var10. Fitted glucoamylase concentration was found from linear regression of absorbances obtained with inhibitor concentrations from 0 to 1 μM. With 2 and 4 μM inhibitor glucoamylase activity was essentially totally inhibited and average of absorbances obtained with these inhibitor concentrations was assumed to correspond to the background of the assay. As 20 μl inhibitor was mixed with 40 μl culture supernatant this resulted in a fitted glucoamylase concentration in the culture supernatant of 0.69 μM and a specific activity on pNP-Glu of 52 mOD/min/μM

Specific activity on pNP-Glu was given as negative of slope of activity as function of inhibitor concentration. Results for the determined concentrations and specific activities are given in Table 2. Standard deviation for determined specific activities was on average 7%.

TABLE 2
Determined concentrations and specific activities of 32 glucoamylase
variants. The results are shown as average ± standard
deviation of three wells with same variant.

| | Concentration | Specific activity |
|---|---|---|
| | (μM) | (mOD/min/μM) |
| WT | 0.27 ± 0.05 | 40 ± 1 |
| Var1 | 0.33 ± 0.01 | 38 ± 1 |
| Var2 | 0.42 ± 0.02 | 30 ± 1 |
| Var3 | 0.4 ± 0.01 | 33 ± 2 |
| Var4 | 0.36 ± 0.02 | 47 ± 4 |
| Var5 | 0.35 ± 0.03 | 48 ± 4 |
| Var6 | 0.47 ± 0.1 | 51 ± 6 |
| Var7 | 0.36 ± 0.01 | 42 ± 4 |
| Var8 | 0.3 ± 0.02 | 57 ± 3 |
| Var9 | 0.32 ± 0.01 | 57.5 ± 0.3 |
| Var10 | 0.71 ± 0.02 | 53 ± 1 |
| Var11 | 0.38 ± 0.01 | 49.2 ± 0.3 |
| Var12 | 0.32 ± 0.04 | 45 ± 4 |
| Var13 | 0.37 ± 0.03 | 41 ± 4 |
| Var14 | 0.36 ± 0.02 | 41 ± 3 |
| Var15 | 0.39 ± 0.05 | 40 ± 3 |
| Var16 | 0.33 ± 0.01 | 48 ± 3 |
| Var17 | 0.3 ± 0.01 | 47 ± 3 |
| Var18 | 0.31 ± 0.02 | 43 ± 0 |
| Var19 | 0.38 ± 0.06 | 49 ± 8 |
| Var20 | 0.38 ± 0.03 | 47.2 ± 0.8 |
| Var21 | 0.35 ± 0.01 | 62 ± 1 |
| Var22 | 0.33 ± 0.01 | 58 ± 2 |
| Var23 | 0.31 ± 0.01 | 62 ± 2 |
| Var24 | 0.33 ± 0.03 | 43 ± 2 |
| Var25 | 0.33 ± 0.02 | 58 ± 3 |
| Var26 | 0.39 ± 0.03 | 48 ± 2 |
| Var27 | 0.35 ± 0.04 | 48 ± 6 |
| Var28 | 0.47 ± 0.06 | 49 ± 4 |
| Var29 | 0.38 ± 0.04 | 56 ± 8 |
| Var30 | 0.41 ± 0.02 | 58 ± 3 |
| Var31 | 0.39 ± 0.03 | 62 ± 4 |

### Example 2

Active Site Titration of Xylanase

The synthesized burst active site titrant 2,4-dinitrophenyl 2-deoxy-2-flouro-β-D-xylopyranoside was dissolved in Milli Q water. 100 μl dissolved titrant was mixed with 50 μl assay buffer (50 mM sodium acetate, 0.0225% Brij 35, pH 5.0) and 50 μl of a purified sample of the commercial xylanase Shearzyme (available from Novozymes) diluted in Milli Q water in the wells of a microtiter plate. Final concentrations of 2,4-dinitrophenyl 2-deoxy-2-flouro-β-D-xylopyranoside were 0.5 mM and 1 mM, whereas the xylanase was added to give final absorbances at 280 nm of 13.4, 6.7 and 3.3. After mixing, absorbance was read at 405 nm at room temperature every 7 min for 30 hours using a SpectraMax Plus (Molecular Devices) microtiter plate reader. After subtraction of absorbances read in wells with same concentration of titrant but without enzyme, the measurements were fitted to the equation:
A₄₀₅=B*(1−exp(−(t+LT)*ln(2)/T_{1/2}))+S*(t+LT)

where A₄₀₅ is the absorbance at 405 nm, B is the burst in absorbance at 405 nm, t is the time from first measurement of absorbance, LT is the lag time from mixing of the reagents to first measurement of absorbance, T_{1/2} is the half time for the exponential burst phase and S is the slope due to hydrolysis of the enzyme 2-deoxy-2-fluoro-β-D-xylopyranose complex.

To calculate the xylanase concentration corresponding to a given absorbance burst, a standard curve obtained from absorbances at 405 nm with known concentrations of 2,4-dinitrophenol in the same volume and buffer was included.

From the results in Table 3 it is seen that hydrolysis of the enzyme 2-deoxy-2-fluoro-β-D-xylopyranose complex (Slope S) is very slow compared to the initial complex formation liberating 2,4-dinitrophenol (T_{1/2}). Furthermore, the xylanase concentrations calculated from the bursts are close to the ones expected from A280 if the enzyme sample was entirely pure and fully active being 152 μM, 76 μM and 38 μM with a theoretical molar extinction coefficient of 87870 M⁻¹ cm⁻¹

TABLE 3
Burst active site titration of the xylanase Shearzyme with the titrant
2,4-dinitrophenyl 2-deoxy-2-flouro-β-D-xylopyranoside.

| | | | | | | |
|---|---|---|---|---|---|---|
| Xylanase A₂₈₀ | 13.4 | 6.7 | 3.3 | 13.4 | 6.7 | 3.3 |
| Titrant (mM) | 1 | 1 | 1 | 0.5 | 0.5 | 0.5 |
| Burst B (A₄₀₅) | 0.90 | 0.48 | 0.23 | 0.84 | 0.47 | 0.24 |
| T_{1/2} (h) | 4.4 | 4.2 | 4.2 | 8.7 | 8.6 | 8.1 |
| Slope S (A₄₀₅/h) | 0.00 | 0.00 | 0.00 | 0.00 | 0.00 | 0.00 |
| Xylanase | 137 | 73 | 35 | 128 | 71 | 37 |
| concentration (μM) | | | | | | |

### Example 3

Synthesis of 2,4-DNP 2-deoxy-2-fluoro-β-D-xylopyranoside

¹H NMR spectra were recorded on a Varian Mercury 400 MHz at 30° C. Flash chromatography was accomplished using a FLASH 40i chromatography module from Biotage. All solvents were purchased from Merck.

### 3,4-di-O-Acetyl-2-deoxy-2-fluoro-β-D-lyxo- and xylopyranosides 1a,b (ASU 14850-046)

3,4-Di-O-acetyl-D-xylal (0.87 g, 4.4 mmol) was dissolved in DMF/H₂O (3:1, 40 mL) and Selectflour (2.5 g, 7.0 mmol) was added. The solution was stirred overnight at room temperature before concentrated. The residue was dissolved in EtOAc (200 mL) and extracted with water (2×50 mL). The aqueous phase was washed with EtOAc (50 mL) and the pooled organic phases were dried with MgSO4, filtered and concentrated to give 0.76 g of crude 1a,b.

### 2,4-DNP 2-deoxy-2-fluoro-α,β-D-lyxo and xylopyranosides 2a,b,c (ASU 14850-049)

The crude product 1a,b (0.40 g, 2.0 mmol) was dissolved in DMF (3 mL) and 2,4-dinitrofluorobenzene FDNB (0.40 g, 2.1 mmol) was added (syringe!) followed by addition of DABCO (1,4-diazabicyclo[2.2.2]-octane, 0.68 g, 6.0 mmol). The solution was stirred overnight and concentrated. The residue was taken into CHCl₃ (100 mL) and extracted with water (2×50 mL) before dried (MgSO₄) and concentrated to give 0.70 g of crude oil. Chromatography (EtOAc/heptane 1:2) gave first pure β-D-lyxo derivative 2b (0.14 g) and second 0.30 g of a mixture of 2a,c. The α-D-lyxo derivative 2c (51 mg) was crystallized from the mixture by addition of cold EtOAc/heptane (1:2).

2b: ¹H NMR (CDCl₃): 8.75 ppm (d, 1H, DNP), 8.45 ppm (dd, 1H, DNP), 7.50 ppm (d, 1H, DNP), 5.90 ppm (d, 1H, J=5 Hz, H-1), 5.54 ppm (m, 1H, H-3), 5.05 ppm (dt, 1H, J=5 and 42 Hz, H-2), 5.03 ppm (m, 1H, H-4), 4.23 ppm (dd, 1H, J=2 and 13 Hz, H-5a) and 3.81 ppm (dd, 1H, J=13 and 1 Hz, H-5b), 2.3 ppm (s, 3H, OAc), 2.2 ppm (s, 3H, OAc).

2c: ¹H NMR (CDCl₃, selected data): 8.83 ppm (d, 1H, DNP), 8.48 ppm (dd, 1H, DNP), 7.55 ppm (d, 1H, DNP), 5.90 ppm (dd, 1H, J=6 and 3 Hz, H-1), 5.32-5.50 ppm (m, 2H, H-3 and H-4), 5.11 ppm (dt, 1H, J=48 and 2 Hz, H-2), 4.10 ppm (dd, 1H, J=11 and 5.5 Hz, H-5a), 3.74 ppm (t, 1H, J=11 Hz, H-5b), 2.2 ppm (s, 3H, OAc, 2.1 ppm (2.1 ppm (s, 3H, OAc).

### 2,4-DNP 2-Deoxy-2-fluoro-β-D-xylopyranoside 3a (ASU 14850-058B)

The mother liquor from the crystallization of 2c containing mainly 2a was deacetylated in 5% HCl—MeOH (prepared by addition of 0.5 mL acetyl chloride to 10 mL MeOH) at room temperature overnight. The solution was concentrated and evaporated from diethylether (25 mL). The β-D-xylo derivative 3a (27 mg) was selectively crystallized from MeOH/diethylether/petroleum ether. Mp. 164-165° C. ¹H NMR (CD₃OD, selected data): J_{2,F}=52 Hz.

### 2,4-DNP 2-Deoxy-2-fluoro-β-D-lyxopyranoside 3b and 2,4-DNP 2-Deoxy-2-fluoro-α-D-lyxopyranoside 3c (ASU

The two pure lyxo-derivatives were deacetylated as described above to give the unprotected 3b and 3c.

3b: ¹H NMR (CD₃OD, selected data): 5.84 ppm (dd, 1H, H-1), 4.95 ppm (dt, 1H, J_{2,F}=54 Hz, H-2).

3c: ¹H NMR (CD₃OD, selected data): 6.20 ppm (dd, 1H, H-1), 4.88 ppm (dt, 1H, J_{2,F}=48 Hz, H-2).

1-9. (canceled)

10. A method for determining the concentration of a glycosyl hydrolase by active site titration using an inhibitor having a Kd which is at least 25 times lower than the concentration of glycosyl hydrolase or, when the glycosyl hydrolase is a retaining glycosyl hydrolase, using a substrate wherein the rate constant for the glycosylation step is at least 10 times larger than for the deglycosylation step.

11. The method of claim 10, wherein Kd is at least 100 times lower than the concentration of glycosyl hydrolase.

12. The method of claim 10, wherein the rate constant for the glycosylation step is at least 100 times larger than for the deglycosylation step.

13. The method of claim 10, wherein the glycosyl hydrolase belongs to family 13 glycosyl hydrolase.

14. The method of claim 10, wherein the glycosyl hydrolase belongs to family 14 glycosyl hydrolase.

15. The method of claim 10, wherein the glycosyl hydrolase belongs to family 15 glycosyl hydrolase.

16. The method of claim 10, wherein the glycosyl hydrolase belongs to family 31 glycosyl hydrolase.

17. The method of claim 10, wherein the glycosyl hydrolase belongs to family 57 glycosyl hydrolase.

18. The method of claim 10, wherein the glycosyl hydrolase belongs to family 63 glycosyl hydrolase.

19. A method of screening for a property of a glycosyl hydrolase wherein the property is dependent on the concentration of the glycosyl hydrolase, comprising the steps of:
a) arranging a population of cells expressing glycosyl hydrolases in a spatial array wherein each position of the spatial array is occupied by one or more cells expressing a specific glycosyl hydrolase,
b) cultivating the cells in a suitable growth medium,
c) determining the concentration of the glycosyl hydrolase of each position of the spatial array by active-site titration using an inhibitor having a Kd which is at least 25 times lower than the concentration of glycosyl hydrolase or, when the glycosyl hydrolase is a retaining glycosyl hydrolase, using a substrate wherein the rate constant for the glycosylation step is at least 10 times larger than for the deglycosylation step,
d) assaying the glycosyl hydrolase of each position of the spatial array for the property and relating the result to the concentration.

20. The method of claim 19, wherein the glycosyl hydrolases are expressed recombinantly by the cells.
